(19) United States
(12) Patent Application Publication
(10) Pub. No.: US 2007/0112649 A1
(43) Pub. Date: May 17, 2007

(54) Material and device inventory tracking system for medical and other uses

(76) Inventor: Kevin Schlabach, Chesterbrook, PA (US)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 11/255,134

(22) Filed: Oct. 20, 2005

Related U.S. Application Data
(60) Provisional application No. 60/620,545, filed on Oct. 20, 2004.

(51) Int. Cl. G06Q 10/00

(52) U.S. Cl. 705/28

(57) ABSTRACT

A system repetitively verifies a quantity of items against a baseline quantity of items, and manages the baseline quantity of items at the same time. A display processor initiates generation of data, representing a display image on a user interface. The display image presents a pre-populated list of item types and associated quantities derived from a first source. The display image also presents individual data fields corresponding to individual item types of the list. An individual data field receives data representing a user entered quantity value representing a count of items of an individual item type. A data processor compares a user entered quantity value with a pre-populated quantity, and generates data representing a message in response to a difference determined by the comparison.

### CROSS-REFERENCE TO RELATED APPLICATIONS

The present application is a non-provisional application of provisional application having Ser. No. 60/620,545 filed by Kevin Schlabach on Oct. 20, 2004.

### FIELD OF THE INVENTION

The present invention generally relates to computer information systems. More particularly, the present invention relates to a material and device inventory tracking system for medical and other uses.

### BACKGROUND OF THE INVENTION

Computer information systems (“systems”) include computers that communicate with each other over a network, such as the Internet, and computers that manage information. The systems may be used to manage inventory control of items. Inventory control includes, for example, identifying, managing, tracking, and reporting the items. Managing the items includes, for example, maintaining supply usage histories, recording present quantities, order quantities, and variances. The items include, for example, individual instruments, sets (e.g., tray, pack, or cart) of instruments, and equipment used in a hospital operating room (OR).

One area of inventory control that can streamline processes in the OR is a physician preference card. A physician preference card provides a description of items requested by a surgeon in the OR for a particular patient surgery. Typically, the surgeon completes the physician preference card. Physician preference cards make it easier for operating room personnel to provide the correct supplies and equipment requested by surgeons for each surgery, thereby limiting the amount of supplies opened and not used. Preference cards may be used to help count a quantity of the items, which are listed on the card. Based on the preference cards, the system generates a pick list that is used to prepare a complete surgical pack or cart that is delivered to the OR for use during a patient's surgery. Inventory control of items before, during, and/or after a patient's surgery is especially important in the OR to ensure that no unintended items are mistakenly left inside a patient's body after surgery.

The systems may be used to track parameters over time. FIG. 1 illustrates a user interface display 100 presenting columnar history, which permits a user of the system to monitor multiple values 102, such as a patient's temperature, across multiple columns 104 that represent a timeline. FIG. 1 does not include inventory control features.

The systems may be used to automate inventory control of items 204 for the OR. FIG. 2 illustrates a user interface display 200 presenting pre-populated data input. The system of FIG. 2 receives the pre-populated data 202, representing OR items, for example, from a preference card, pick list, or other document.

FIG. 3 illustrates a user interface display 300 presenting delta data input 304 to permit the user to enter quantity inputs of items 302 in an OR. The delta data input includes an input to change the quantity of an item, and an indication of whether the change in the quantity is an addition or subtraction to the total quantity, thereby permitting the system to calculate a new total quantity.

Other systems (not shown) provide electronic count sheets limited to a simple count that is entered in a single manner similar to a paper count sheet.

Still other systems (not shown) permit a user to enter quantity inputs by hash marks in a hash table, grid, or form, wherein each marked box in the grid represents an item counted. Some hospitals implement hash tables on paper for count sheets or other documentation forms, such as, for example, a flow sheet used by an anesthesiologist when monitoring medications and vitals across time.

Existing systems fail to provide comprehensive inventory control of items, such as in a critical environment, like an OR, for example. Accordingly, there is a need for a material and device inventory tracking system for medical and other uses that improves over the prior systems.

### SUMMARY OF THE INVENTION

A comprehensive inventory control system, which supports validating a medical device and material inventory, includes a display processor and a data processor. The display processor initiates generation of data, representing a display image on a user interface. The display image presents a pre-populated list of item types and associated quantities derived from a first source. The display image also presents individual data fields corresponding to individual item types of the list. An individual data field receives data representing a user entered quantity value representing a count of items of an individual item type. The data processor compares a user entered quantity value with a pre-populated quantity, and generates data representing a message in response to a difference determined by the comparison.

### BRIEF DESCRIPTION OF THE DRAWINGS

FIG. 1 illustrates a user interface display presenting columnar history, in accordance with the prior art.

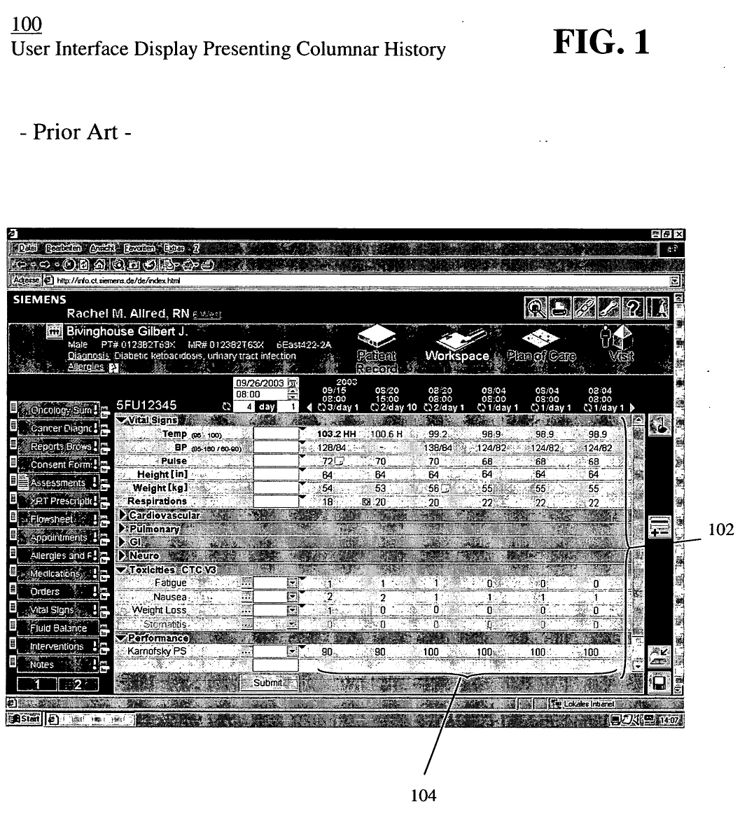

FIG. 2 illustrates a user interface display presenting pre-populated data input, in accordance with the prior art.

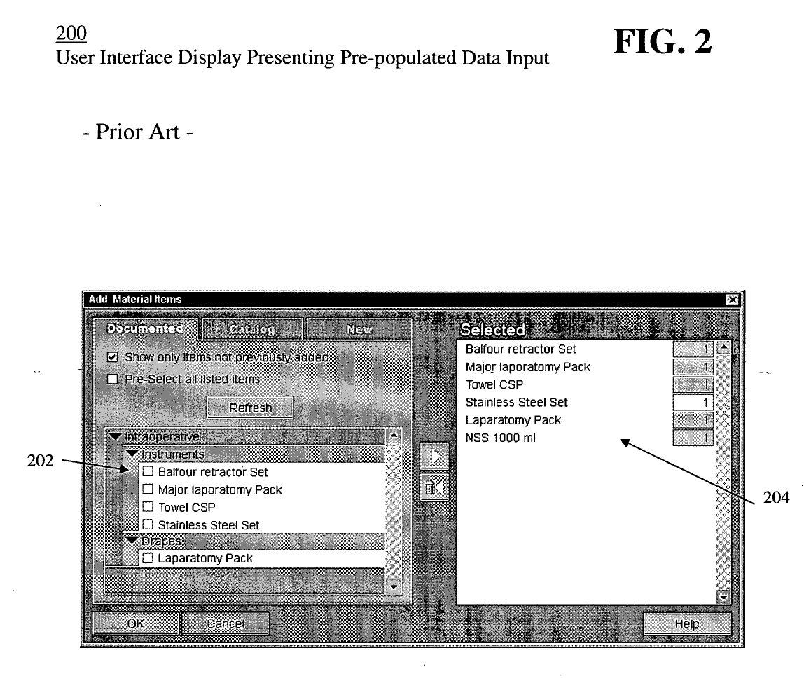

FIG. 3 illustrates a user interface display presenting delta data input, in accordance with the prior art.

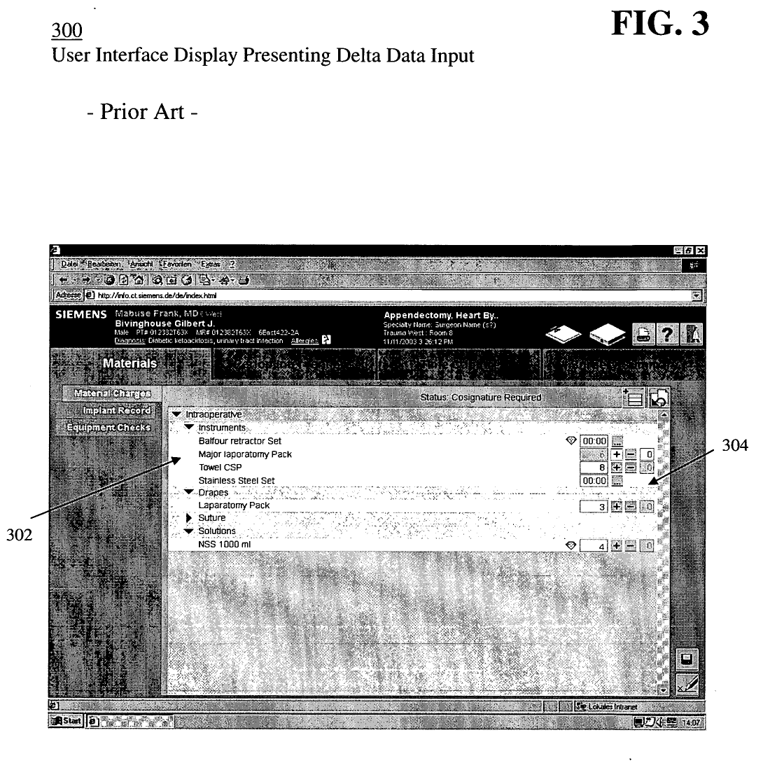

FIG. 4 illustrates a system for material and device inventory control, in accordance with invention principles.

FIG. 5 illustrates a method for material and device inventory control, in accordance with invention principles.

FIG. 6 illustrates a user interface display presenting a base count, in accordance with invention principles.

FIG. 7 illustrates a user interface display presenting hash table counting tool, in accordance with invention principles.

FIG. 8 illustrates a user interface display presenting a dialog box to change item list by importing from previous documentation, in accordance with invention principles.

FIG. 9 illustrates a user interface display presenting a dialog box to change item list by importing from an inventory catalog, in accordance with invention principles.

FIG. 10 illustrates a user interface display presenting a dialog box to change item list by direct user entry, in accordance with invention principles.

### DETAILED DESCRIPTION OF THE PREFERRED EMBODIMENTS

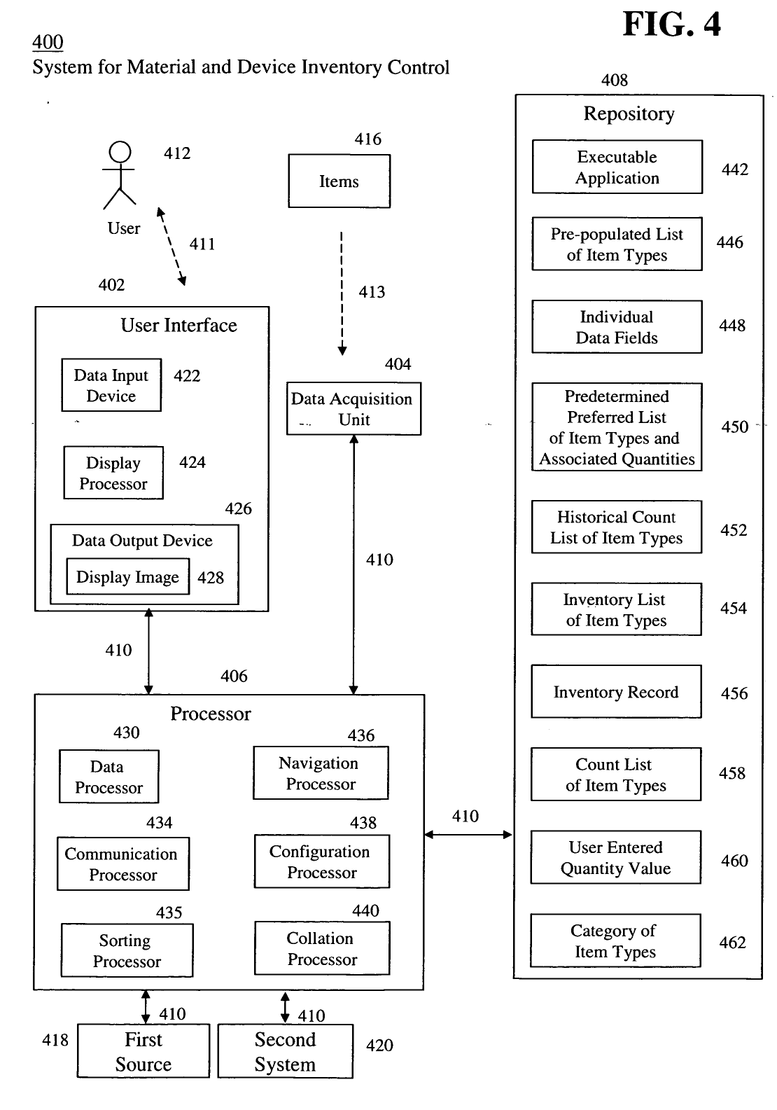

FIG. 4 illustrates a system 400 for material and device inventory control. The system 400 includes a user interface 402, a data acquisition unit 404, a processor 406, and a repository 408. A user 412, items 416, a first source 418, and a second system 420 interface and/or interact with the system 400.

A communication path 410 interconnects elements of the system 400, and/or interconnects the system with the first source 418 and/or the second system 420. The dotted line near reference number 411 represents interaction between the user 412 and the user interface 402. The dotted line near reference number 413 represents interaction between the items 416 and the data acquisition unit 404.

The user interface 402 further provides a data input device 422, a display processor 424, and a data output device 426. The data output device 426 further includes one or more display images 428.

The processor 406 further includes a data processor 430, a communication processor 434, a sorting processor 435, navigation processor 436, a configuration processor 438, and a collation processor 440.

The repository 408 further includes an executable application 442, a pre-populated list of item types 446, individual data fields 448, a predetermined preferred list of item types and associated quantities of items 450, a historical count list of item types 452, an inventory record 456, a count list of item types 458, a user entered quantity value 460, and a category of item types 462.

The system 400 may be employed by any type of enterprise, organization, or department, such as, for example, providers of healthcare products and/or services responsible for servicing the health and/or welfare of people in its care. For example, the system 400 represents a hospital information system. A healthcare provider provides services directed to the mental, emotional, or physical well being of a patient. Examples of healthcare providers include a hospital, a nursing home, an assisted living care arrangement, a home health care arrangement, a hospice arrangement, a critical care arrangement, a health care clinic, a physical therapy clinic, a chiropractic clinic, a medical supplier, a pharmacy, and a dental office. When servicing a person in its care, a healthcare provider diagnoses a condition or disease, and recommends a course of treatment to cure the condition, if such treatment exists, or provides preventative healthcare services. Examples of the people being serviced by a healthcare provider include a patient, a resident, a client, and an individual.

The system 400 may be fixed and/or mobile (i.e., portable), and may be implemented in a variety of forms including, but not limited to, one or more of the following: a personal computer (PC), a desktop computer, a laptop computer, a workstation, a minicomputer, a mainframe, a supercomputer, a network-based device, a personal digital assistant (PDA), a smart card, a cellular telephone, a pager, and a wristwatch. The system 400 and/or elements contained therein also may be implemented in a centralized or decentralized configuration. The system 400 may be implemented as a client-server, web-based, or stand-alone configuration. In the case of the client-server or web-based configurations, one or more of the executable applications 442 may be accessed remotely over a communication network.

The communication path 410 (otherwise called network, bus, link, connection, channel, etc.) represents any type of protocol or data format including, but not limited to, one or more of the following: an Internet Protocol (IP), a Transmission Control Protocol Internet protocol (TCPIP), a Hyper Text Transmission Protocol (HTTP), an RS232 protocol, an Ethernet protocol, a Medical Interface Bus (MIB) compatible protocol, a Local Area Network (LAN) protocol, a Wide Area Network (WAN) protocol, a Campus Area Network (CAN) protocol, a Metropolitan Area Network (MAN) protocol, a Home Area Network (HAN) protocol, an Institute Of Electrical And Electronic Engineers (IEEE) bus compatible protocol, a Digital and Imaging Communications (DICOM) protocol, and a Health Level Seven (HL7) protocol.

The system 400, elements, and/or processes contained therein may be implemented in hardware, software, or a combination of both, and may include one or more processors, such as processor 406. A processor is a device and/or set of machine-readable instructions for performing task. The processor includes any combination of hardware, firmware, and/or software. The processor acts upon stored and/or received information by computing, manipulating, analyzing, modifying, converting, or transmitting information for use by an executable application or procedure or an information device, and/or by routing the information to an output device. For example, the processor may use or include the capabilities of a controller or microprocessor.

The user interface 402 permits bidirectional exchange of data between the system 400 and the user 412 of the system 400 or another electronic device, such as a computer or application.

The data input device 422 typically provides data to a processor in response to receiving input data either manually from a user or automatically from an electronic device, such as a computer. For manual input, the data input device is a keyboard and a mouse, but also may be a touch screen, or a microphone with a voice recognition application, for example.

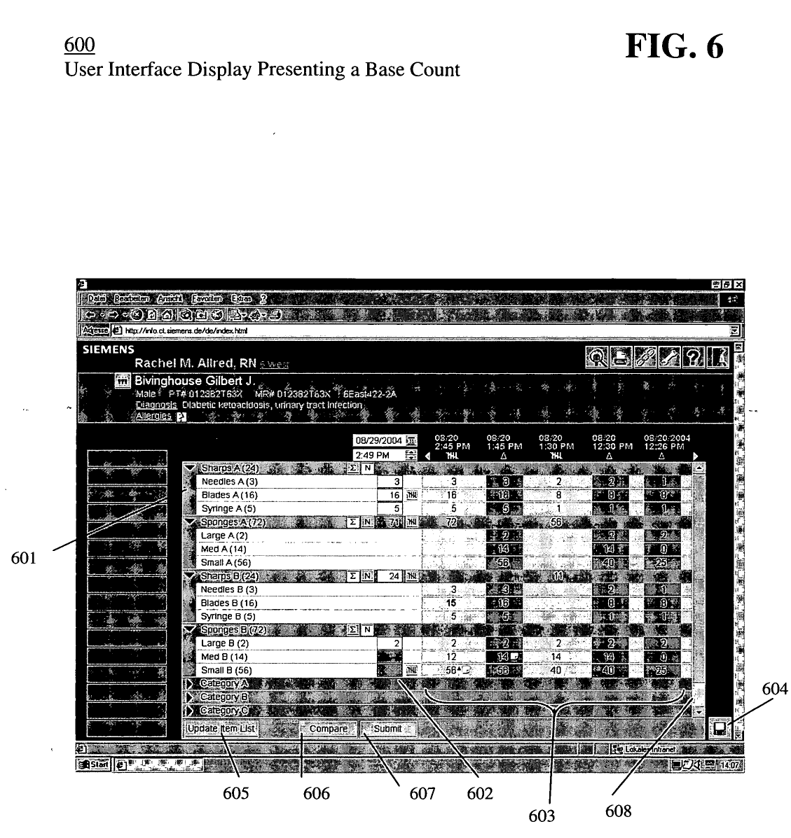

The data output device 426 typically provides data from a processor for use by a user or an electronic device or application. For output to a user, the data output device 426 is a display, such as, a computer monitor (e.g., screen), that generates one or more display images 428 in response to receiving the display signals from the display processor 424, but also may be a speaker or a printer, for example. FIGS. 6-10 illustrate examples of the display images 428.

The user-interface display image 428 includes image elements enabling the user 412 to enter data representing a user entered quantity value representing the count of items of the individual item type in multiple different ways, as shown in FIG. 6-10. The multiple different ways include one or more of the following: (a) by entry of a numerical value, (b) by selection of a decrement or increment button, (c) by entry of data indicating a group of multiple items, and (d) by selection from a predetermined option list.

The display processor 424 or generator includes electronic circuitry or software or a combination of both for generating display images or portions thereof. The data output device 426, implemented as a display, is coupled to the display processor 424 and displays the generated display images. The display images 428 permit user interaction with the processor 406 or other device. The display processor 424 may be implemented in the user interface 402 and/or the processor 406.

The repository 418 represents any type of storage device, such as computer memory devices or other tangible storage medium. The repository 418 represents one or more memory devices, located at one or more locations, and implemented as one or more technologies, depending on the particular implementation of the system 400. The repository includes data representing multiple count lists of item types 458 (see 601 and 602 in FIG. 6) previously entered by the user 412 via the user-interface display image 428 together with dates of creation of individual lists. The navigation processor 436 enables the user 412 to access and display selected count lists 458 derived from the repository 408.

The configuration processor 438 enables a user to associate a particular item type with a particular category or group of item types 462, which may include a subset of items. The collation processor 440 automatically allocates received data representing the user entered quantity value, representing the count of items of the individual item type, to a particular category. The sorting processor 435 automatically enters received data, representing quantity values representing counts of the subset of items, in response to user entry of data representing a count of the particular item.

The executable application 442 comprises machine code or machine readable instruction for implementing predetermined functions including, for example, those of an operating system, a software application program, a healthcare information system, or other information processing system, for example, in response to user command or input. An executable procedure is a segment of code (i.e., machine readable instruction), sub-routine, or other distinct section of code or portion of an executable application for performing one or more particular processes, and may include performing operations on received input parameters (or in response to received input parameters) and providing resulting output parameters. A calling procedure is a procedure for enabling execution of another procedure in response to a received command or instruction. An object comprises a grouping of data and/or executable instructions or an executable procedure.

The data acquisition unit 404 provides an input mechanism for identifying the items and for updating count values in individual data fields, as shown in FIG. 6, in response to the received data identifying the items. The data acquisition unit 404 may employ any technology including, for example, wireless identification, such as radio frequency (RF) and optical technology, for example.

RF identification technology example employs an RF reader in the data acquisition unit 404 and an RF identification (ID) tag carried with the item. The RFID tag uniquely identifies an item using an electronic identification, such as, for example, a radio frequency or code. When a user moves the RF reader in close proximity to the RFID tag carried with the item, the RF reader detects the electronic identification of the RFID tag. The data acquisition unit 404 sends the detected electronic identification of the RFID tag to the processor 406. The processor 406 matches the electronic identification of the RFID tag to a description of the associated item stored in the repository 408, and records the match in the repository 408.

The processor 406 uses the recorded information to automatically pre-populate or populate an electronic count sheet, as shown in FIG. 6. For example, in an OR, when items are moved within close proximity of the operating table, the system 400 automatically prompts the user to add the detected items to the count sheet baseline. When items are brought into the operating room during the surgery, the system 400 automatically prompts the user to add the items to the count sheet baseline.

Examples of items having RFID tags and used in an OR include, for example, surgical instruments, such as clamps, retractors, scissors, and scalpels, and single use items, such as sponges, towels, gauze, dressing tape rolls, and other surgical errata.

The system 400 also permits a user to automatically locate items contained on the count sheet that the user cannot find when performing a count in the OR before, during, or after surgery. The user selects one or more items on the count sheet, as shown in FIG. 6, and activates the system 400 to locate the selected items (e.g., location within room, on a given table, in an area of the patient).

For example, a surgical table may carry or be proximate to one or more RFID readers that periodically or constantly identify the location of tagged items. The system 400 automatically initializes the count procedure before a surgical procedure by identifying the inventory located within range of the surgical table. Periodically during the surgical procedure, the system 400 reports the location of tagged items: unused items on the sterilized carts or trays, used items on the contaminated carts or trays, items on the operating table, and/or items that are internal to a patient's anatomy.

The system 400 alerts the surgical team with either blinking screen or audio alert, for example, of tagged items that remain within the patient. The system 400 also warns when items are not at any of the known locations (e.g., sterile cart, contaminated cart, table), allowing the surgical team to determine the status of a missing item. The system's count function may be initiated, for example, by an audio command or by pressing a foot pedal or other user command.

Various forms of RFID tags, such as active or passive tags, may be used depending on the particular application, such as size, cost, reliability, etc. For example, in an OR environment, the RFID tags, as well as the associated items, are designed to be robust and rugged enough to withstand a sterilization process that reduces or prevents infection of a patient during surgery, without negatively affecting the RFID tags.

Optical technology, for example, employs an optical reader or scanner in the data acquisition unit 404 and an optical identification (ID) tag carried with the item. The optical ID tag (e.g., bar code) uniquely identifies an item using a machine-readable representation of information in a visual format on a surface of the item. Bar codes may store data in the widths and spacings of printed parallel lines, patterns of dots, concentric circles, or hidden in images. When a user moves the optical reader in close proximity to the optical ID tag on the item, the optical reader detects the visual identification of the optical ID tag. The data acquisition unit 404 sends the detected visual identification of the optical ID tag to the processor 406. The processor 406 matches the visual identification of the optical ID tag to a description of the associated item stored in the repository 408, and records the match in the repository 408. Bar codes are used to implement Auto ID Data Capture (AIDC) systems that improve the speed and accuracy of computer based inventory control.

For example, a user uses the optical scanner to automatically add or remove items counted in the OR before, during, or after surgery from the count sheet baseline, as shown in FIG. 6. Hence, the optical example provides similar advantages as the RF example for inventory control. However, the optical example uses an optical path between the optical reader and the optical ID tag; whereas, the RF example uses a RF path between the RF reader and the RFID tag.

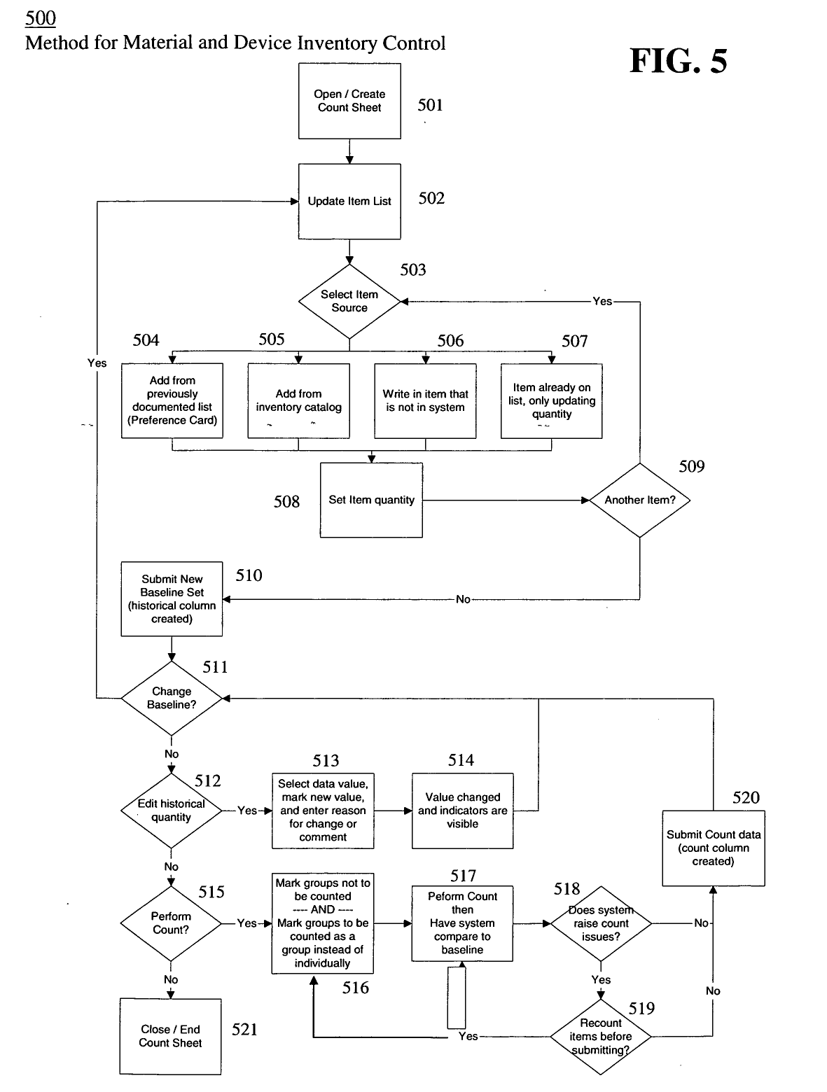

FIG. 5 illustrates a method 500 for material and device inventory control. The method 500 provides a workflow for using the electronic count sheet. Any portion or the entire method 500 or variations thereof may be employed in a tangible storage medium (i.e., the repository 408) incorporating machine-readable instructions (e.g., executable application 442) for performing the activities of the method 500.

At step 501, the system 400 opens a count sheet, as shown in FIG. 6, responsive to a user's manual input or to the system's 400 automatic input, for example. The display processor 424 initiates generation of data representing a user-interface display image 428 presenting the pre-populated list of item types 446 and the individual data fields 448. The pre-populated list of item types 446 and associated quantities are derived from the first source 418. The individual data fields 448 correspond to individual item types of the pre-populated list of item types 446. An individual data field is for receiving data representing a user entered quantity value representing a count of items of an individual item type.

At step 502, the system 400 updates a list of items responsive to the system's determination to change an item baseline count at step 511.

At step 503, the system 400 selects a source of items from which to import the items to the system 400 responsive to a user's manual input or to the system's 400 automatic input, for example.

The pre-populated list of item types 446 and associated quantities are derived from multiple different sources including the first source 418 and are editable by the user 412. The multiple different sources includes two or more of the following: (a) a predetermined preferred list of item types 450 to be used for a particular procedure by a healthcare worker, (b) a predetermined preferred list of item types 450 to be used for operation of a particular medical device type, (c) a predetermined inventory list of item types, (d) a historical count list of item types 452 previously entered by a user via the user interface display image 428 derived from the repository 408, and (e) an inventory list of item types 454 imported from the second system 420.

The imported items provide a baseline count of the items for the system 400. The data processor 430 compares a user entered quantity value 460 with a pre-populated quantity derived from a selected count list of the multiple count lists of item types 458, and generates data representing a message in response to a difference determined by the comparison.

At step 504, the system 400 adds a description of an item from a previously documented list of items (e.g., a preferred card). For example, the user 412 pulls items from documented sources, as needed.

At step 505, the system 400 adds a description of an item from an inventory catalog. For example, the user 412 pulls items from catalog sources, as needed.

At step 506, the system 400 receives a description of an item that is not in the system 400 responsive to a user's manual input or to the system's 400 automatic input, for example. For example, the user 412 writes in items, as needed.

At step 507, the system 400 receives a description of an item already on a known list. Since the description of the item is already known, the system 400 updates the quantity of the item.

At step 508, the system 400 receives a quantity count for the description of the item responsive to a user's manual input or to the system's 400 automatic input, for example. The communication processor 434 automatically updates inventory records 456 in the repository 408 in response to the quantity value entered by the user or imported by the system 400.

At step 509, the system 400 determines whether a description of another item is to be entered into the system 400 responsive to a user's manual input or to the system's 400 automatic input, for example. If the determination at step 509 is positive, the method 500 returns to step 503. If the determination at step 509 is negative, the method 500 continues to step 510.

At step 510, the system 400 submits a new baseline set (i.e., a historical column in the count sheet), as shown in column 602 in FIG. 6.

At step 511, the system 400 determines whether to change a baseline responsive to a user's manual input or to the system's 400 automatic input, for example. For example, the user updates the baseline count for one or more items, as needed, throughout surgery on a patient. If the determination at step 511 is positive, the method 500 returns to step 502. If the determination at step 511 is negative, the method 500 continues to step 512.

At step 512, the system 400 determines whether to edit a historical quantity, as shown in columns 603 in FIG. 6, responsive to a user's manual input or to the system's 400 automatic input, for example. If the determination at step 512 is positive, the method 500 continues to step 513. If the determination at step 512 is negative, the method 500 continues to step 515.

At step 513, the system 400 selects a new data value, marks a new data value, and enters a reason for the change or a comment. The display image 428 may include individual data fields 448 for receiving data representing a user entered reason code for editing the pre-populated list of item types 446. For example, a user may edit the history to indicate that a sponge was cut into two pieces or that a needle broke in half, and that the item is now counted as two pieces instead of one piece.

At step 514, the system 400 changes the value and displays indicators, representing the new data value, and reason or comment, as shown in columns 603 next to the quantity counts in FIG. 6.

At step 515, the system 400 determines whether to perform a count responsive to a user's manual input or to the system's 400 automatic input, for example. For example, the user 412 may choose to perform counts, as needed, throughout surgery on a patient. If the determination at step 515 is positive, the method 500 continues to step 516. If the determination at step 515 is negative, the method 500 continues to step 521.

At step 516, the system 400 marks groups not to be counted responsive to a user's manual input or to the system's 400 automatic input, for example. For example, the user 412 collapses (i.e., closes) categories of items that the user does not want to count during the recount. The system 400 also marks groups to be counted as a group instead of individually. For example, the user 412 may select categories or groups of items that include multiple individual items to count the items in the same category. Hence, a user may perform full or partial counts, or may perform counts on some items (e.g., sharps (i.e., sharp items used in the OR)) more frequently than others.

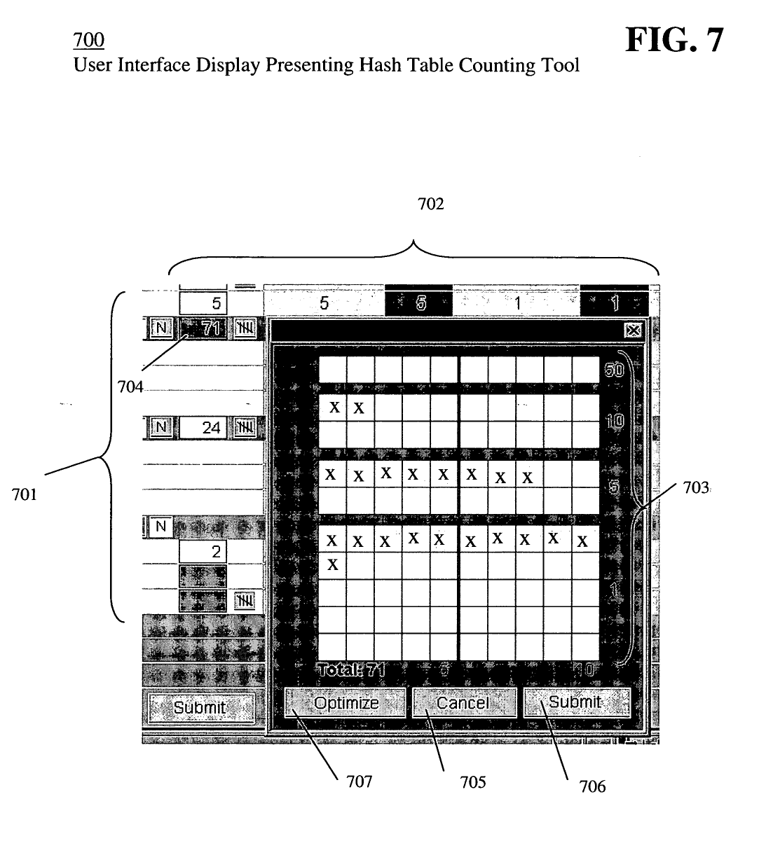

At step 517, the system 400 performs the present count responsive to a user's manual input or to the system's 400 automatic input, for example. For items that have an expected count value above the system's preference count value, the user may use the hash table counting tool, as shown in FIG. 7, to account for the difference in the count values. The system 400 then compares the present count to the baseline count responsive to a user's manual input or to the system's 400 automatic input, for example. For example, after the user selects (e.g., clicks with a mouse) a “compare” input button 606, the system 400 responds by marking all empty fields in expanded categories with warning color, and marks all count entries that don't match the baseline count with warning color.

At step 518, the system 400 determines whether the there are any issues related to the count responsive to a user's manual input or to the system's 400 automatic input, for example. For example, the user may decide if steps 516 and 517 need to be performed again based on the system's response to the comparison performed in step 517. If the determination at step 518 is positive, the method 500 continues to step 519. If the determination at step 518 is negative, the method 500 continues to step 520.

At step 519, the system 400 determines whether to recount items before submitting the count data to the count sheet responsive to a user's manual input or to the system's 400 automatic input, for example. If the determination at step 519 is positive, the method 500 returns to step 516. If the determination at step 519 is negative, the method 500 continues to step 520.

At step 520, the system 400 submits the count data to the count sheet, and a count history column in the count sheet is created or updated.

At step 521, the system 400 closes (i.e., ends) the count sheet responsive to a user's manual input or to the system's 400 automatic input, for example. At any time during the method the count sheet may be saved responsive to a user's manual input or to the system's 400 automatic input, for example.

The method 500 employed by the system 400 advantageously improves efficiency and manages the organization of items, stores the baselines of quantities, converts inventory into countable items, maintains historical documentation, standardizes documentation, and handles count problem notifications. The method 500 removes these tasks from being performed by a user. The method 500 improves usability by using a low number of user interface screens (i.e., display images), by providing multiple interaction options to a user, and by adapting to the needs of an individual user (e.g., clinician). The method 500 increases patient safety by removing the need for a user to verify count matches across time. The method 500 logically performs this for the user, which increases the accuracy of the count. The method 500 may also separate the task of determining a baseline count of inventory (e.g., steps 510 and 511) from the task of counting inventory (e.g., steps 515-519) to achieve the same functionality, but in a less integrated manner and by using more display screens.

FIG. 6 illustrates a user interface display 600 presenting a base count. The display 600 is presented, for example, within an empty framework of a clinical information system.

The display includes a first column 601, a second column 602, several columns 603 at the right side of the second column 602, a save button 604, an update button 605, a compare button 606, a submit button 607, and a scroll function 608.

The first column 601 includes inventory items listed vertically down the left side with one item per row. The inventory items are grouped into categories that may be expanded into view or collapsed from view. Further, categories may contain categories in a hierarchical fashion.

The second column 602 includes a count for the inventory items listed in the first column 601. The inventory items may be counted for a given point in time. An inventory group has a selection to determine if the count for that group is to be performed per item or for the entire group. For those items being counted (e.g., item or group) where the expected total is over a system-stored preference value, the hash counting function (shown in FIG. 7) may be accessed to assist in performing the count, responsive to a user selecting a button (e.g., an image of five hash marks) next to the form field. The header of the second column 602 allows the user to document the time of count being performed even though the system 400 defaults it with the current time.

The several columns 603 at the right side of the second column 602 displays historical count values for the various categories and items in the first column 601. The system 400 creates a historical update to this count sheet by creating a column. These columns can be scrolled through from left to right using arrow on either side of the column header area. A column has a header identifying a timestamp associated with that column. A column has a header identifying whether the column represents a count performed (e.g., an image of five hash marks) or item baseline change (e.g., an image of a triangle). For columns that represent an item baseline change, each item row displays the new quantity for a particular item. For columns that represent a count, the appropriate rows display the count data entered when the count was submitted. If the system 400 identifies a value as a risk, the system 400 displays data in bold red. If the value has been modified, the system 400 displays an indicator (e.g., an image of an upward pointing arrow). If the value has an associated comment, the system 400 displays an indicator (e.g., image of a paper note).

The save button 604 on the right allows the user to save a version of the count sheet to the system 400.

The update button 605 at the bottom provides access to change the baseline list and quantities of the items being counted.

The compare button 606 at the bottom provides access to a system check of the count values against the baseline to identify count issues before submitting the count into a history column.

The submit button 607 at the bottom saves the entered count data into the history of the count sheet, creating a history column.

The scroll function 608 permits the entire display area to be scrollable, without losing visibility of the column headers or footer row containing the buttons 605-607.

FIG. 7 illustrates a user interface display 700 presenting hash table counting function. The display 700 is accessible from the base count display 600, as shown in FIG. 6. The user uses the hash table counting function to tabulate a single item quantity total to permit the system 400 to populate the single item quantity total into the form field that the display 700 was triggered from. The display 700 includes a left column 701, a top row 702, a count grid 703, a total count 704, a cancel button 705, a submit button 706, and an optimize button 707.

The left column 701 corresponds to a portion of rows presented in the second column 602 shown in the display 600 of FIG. 6. For example, the numbers “5,” “71,” “24,” and “2” correspond to the counts presented in the second column 602 shown in the display 600 of FIG. 6 for the “Syringe A,” Sponges A,” “Sharps B,” and Sponges B” presented in the first column 601 shown in the display 600 of FIG. 6.

The top row 702 corresponds to the columns presented in the third row shown in the display 600 of FIG. 6. For example, the numbers “5,” “5,” “5,” “1,” and “1” correspond to the latest and historical counts presented in the sec third row shown in the display 600 of FIG. 6 for the “Syringe A.”

The grid 703 of boxes is presented in system-configured denominations (e.g., 1, 5, 10, and 50) of counting units, which are grouped by denomination (e.g., 1, 5, 10, 50). Clicking on a box in the grid 603 by a user marks the box (e.g., with a mark or highlighted with a color). Clicking and dragging across multiple boxes by a user marks the boxes that the user dragged across (e.g., clicking on one box at the beginning of a row and dragging the cursor across three additional boxes marks four total boxes in the same row). Clicking a box within a denomination by a user marks all preceding boxes within the same denomination (e.g., clicking on box three in denomination one, also automatically marks boxes one and two, as well as box three).

The total count 704 is dynamically tallied to clarify the value currently represented in the count grid 703. For example, the count grid 703 includes the denominations (10×2)+(5×8)+(11×1)=71 for the total count 704.

The cancel button 705 closes the display 700 with no change to the form field in the display 600 of FIG. 6.

The submit button 706 populates the new total value into the form field in the display 600 of FIG. 6.

The optimize button 706 invoke an algorithm (e.g., an executable application, software application) to consolidate boxes marked in lower denominations into fewer boxes in higher denominations.

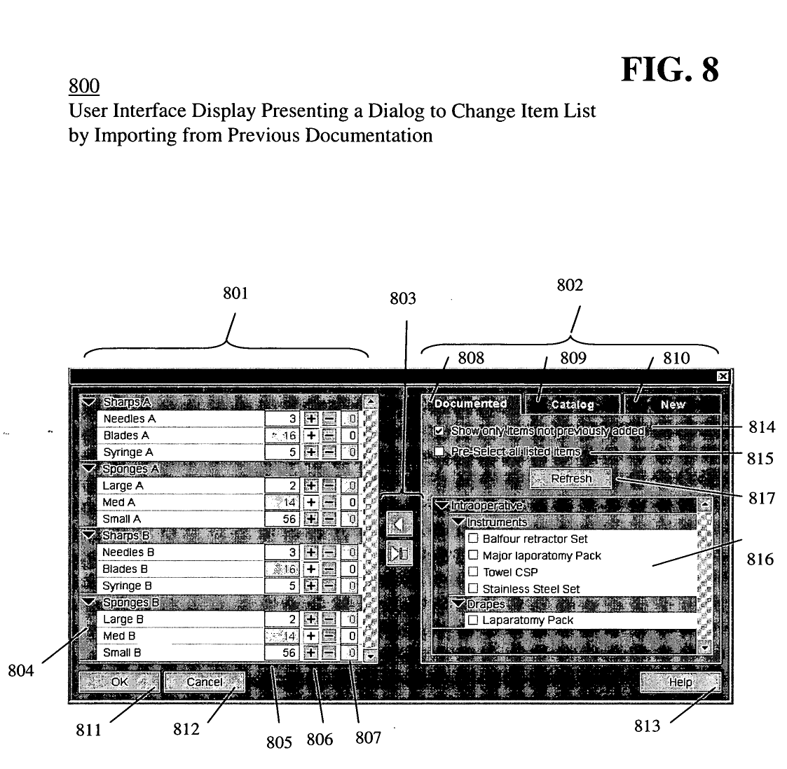

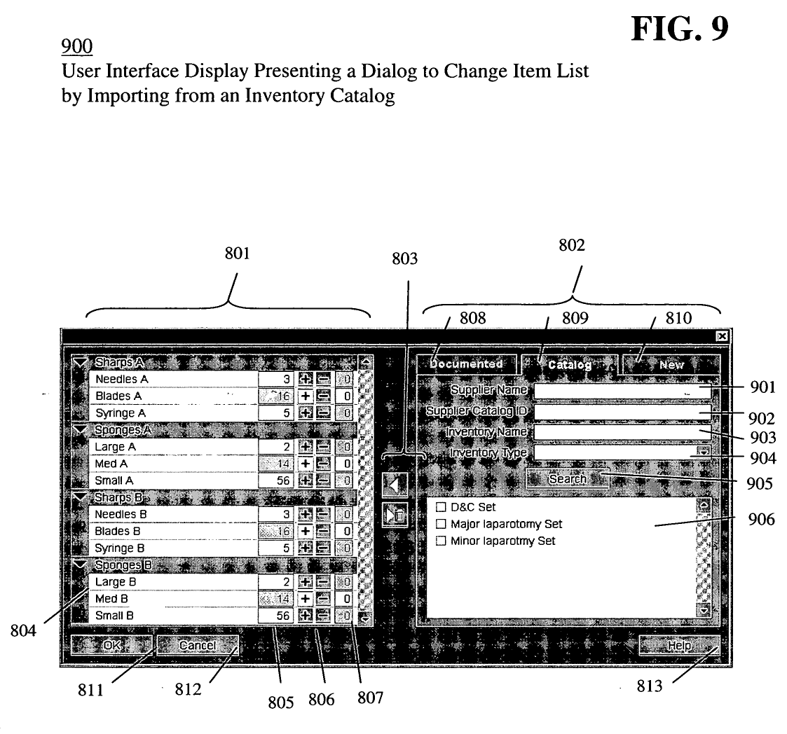

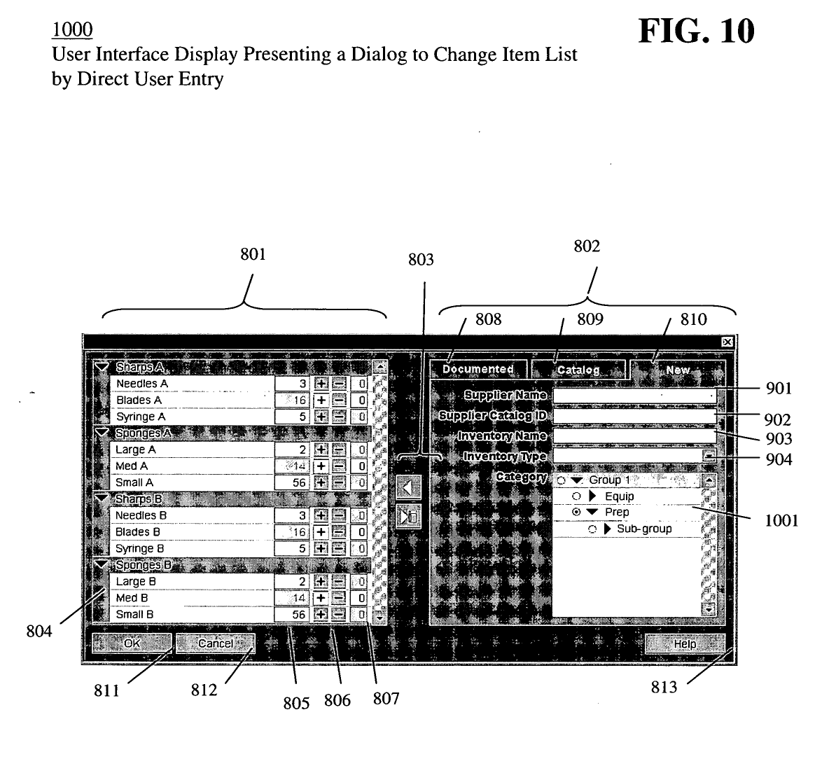

FIG. 8 illustrates a user interface display 800 presenting a dialog to change item list by importing items from previous documentation. FIG. 9 illustrates a user interface display presenting a dialog to change item list by importing items from an inventory catalog. FIG. 10 illustrates a user interface display presenting a dialog to change item list by direct user entry.

The common features of FIGS. 8, 9, and 10 include a left pane 801, a right pane 802, middle buttons 803.

The left pane 801 of each of FIGS. 8, 9, and 10 further includes an inventory of items 804, a count 805 for each item, an increment/decrement adjustor 806, and a count adjustment 807. The left pane 801 contains current item list baseline that the count sheet is using to measure against. The inventory items 804 are listed vertically down the left side, with one item per row. The inventory items 804 are grouped into categories (e.g., “Sharps A,” “Sponges A”) that may be expanded into view or collapsed from view. Categories may also contain sub-categories. The user 412 modifies the quantity for an item using the increment/decrement adjustor 806 by clicking on “+” to increase the quantity by one count per click, or by clicking on “−” to decrease the quantity by one count per click. The user 412 modifies the quantity for an item by entering a new total amount in the count adjustment 807. The quantity that the user enters by either method may be an amount to be added or subtracted from the current count 805.

The right pane 802 of each of FIGS. 8, 9, and 10 further includes tabs indicating sources of the items for importing to the system 400. The tabs include a Documented tab 808, a Catalog tab 809, and a New tab 810.

The middle buttons 803 permit movement of items from one pane 801 or 802 into the other pane 801 or 802, responsive to selection by the user 412 of the right arrow (i.e., left pane 801 to right pane 802) or left arrow (i.e., right pane 802 to left pane 801). For items that are being pulled from the documented tab 808 or the catalog tab 809, the system 400 automatically places those items in the appropriate category in the left pane 801.

Each of FIGS. 8, 9, and 10 also includes an OK button 811, a Cancel button 812, and a Help button 813. The OK button 811 updates the baseline list of items on the count sheet 600, shown in FIG. 6, and associated quantities 805 to match the list of items 804 in the left pane 801. The Cancel button 812 cancels one or more inputs, selections, actions, etc. in FIG. 8, 9, or 10. The Help button 813 provides the user 412 with help or guidance related to one or more inputs, actions, functions, etc. in FIG. 8, 9, or 10.

In particular, FIG. 8 illustrates further details of the Documented tab 808. The Documented tab 808 pulls items from other sources in the system 400 where documentation already contains a list of items (e.g., preference cards). The Document tab 808 further includes a first filter 814, a second filter 815, a list of items 816, and a Refresh button 817. The first filter 814 ensures that items that have not been added to the left pane 801 yet are listed in the list of items 816. The second filter 815 pre-selects multiple (e.g., all) items in the list 816 to save the user time. The list of items 816 contains categories of items including a list of inventory items to be selected. If an item in the list 816 is a set of items, then the system 400 translates the set of items into individual countable items when moved into the left pane 801. The system 400 updates the list of items 816 responsive to the user 412 selecting the Refresh button 817.

In particular, FIG. 9 illustrates further details of the Catalog tab 809. The Catalog tab 809 permits the system 400 to pull items from an inventory catalog sources accessible by the system 400. The system 400 uses one or more item descriptions 901-904, such as supplier name 901, supplier catalog ID 902, inventory name 903, and inventory type 904, each having a field to receive user input to invoke a search of desired items in the inventory catalog. After the user input the desired search terms into the item descriptions 901-904, the system 400 searches items in the inventory catalog responsive to the user selecting the Search button 905. The system 400 displays matching items from the search in a results window 906. The user may select the matching items as a set or individually. If the matching item displayed is a set of items, then the system 400 translates the set of items into the individual countable items when moved into the left pane.

In particular, FIG. 10 illustrates further details of the New tab 810. The New tab 810 is used when the user cannot find the desired item in other sources, so the user 412 manually writes a description of the item. The system 400 uses one or more item descriptions 901-904, such as the supplier name 901, the supplier catalog ID 902, the inventory name 903, and the inventory type 904 to describe the item or groups of items. Each of the item descriptions 901-904 has a field to receive user input to define the desired items. By comparison, the item descriptions 901-904 in FIG. 10 are used to define an item; whereas, the item descriptions 901-904 in FIG. 9 are used to search for an item. The user 412 selects from a category area 1001 an appropriate category corresponding to a category displayed on the count sheet 600, shown in FIG. 6, in which to input the item description.

The system 400 enables a user of an electronic operating room management system to utilize a single and simple set of screens to perform instrument counts within the operating room throughout surgery as needed or requested. The system 400 provides functionality to be used concurrently whenever the end user is performing or managing instrument counts on a count sheet during surgery. The functionality includes: pre-population of items, ability to manage a count baseline, review history of count baseline, perform counts, review the history of counts, input quantities through multiple methods (e.g., total, delta, hash), and increase patient safety by having the system 400 measure and pro-actively communicate counts that don't match the count baseline.

The system 400 (including the method 500) advantageously includes one or more of the following features.

1. The system 400 provides greater access to standardized input sources to add inventory. For example, when adding items, items are pulled from interfaces outside the count sheet such as prior documentation (e.g., preference cards) or inventory catalogs. Items pulled from documentation by default are filtered to ensure a user does not accidentally pull items multiple times creating erroneous data. Items are pulled from sources in the same workflow.

2. The system 400 removes the need for the user to place items into categories on the count sheet. If the input sources are set up appropriately (e.g., preference cards and inventory catalog), then the system automatically places an added item into the appropriate category on the count sheet. For example, a scalpel could dynamically be inserted in the ‘Sharps’ category on a count sheet.

3. The system 400 removes the need for user to convert inventory items to countable items. The system 400 automatically converts an item of multiple parts (e.g., an instrument tray) into the individual countable items to be monitored and counted on the count sheet. An added set is placed onto the count sheet into appropriate categories and items allowing the user to select the single tray, but have the system 400 prompt to count each item in the tray.

4. The system 400 removes need for the user to calculate inventory totals. The user can focus on the items being added and removed from the sterile field, and expect the system 400 to monitor and communicate the baseline of items in the sterile field within the same screen used to perform inventory counts.

5. The system 400 allows user to perform item counts in a flexible manner. For each count being performed, items can be counted as a group or individually. Items can be counted numerically or by hash table (e.g., this is especially valuable for items of high quantity such as sponges). Items for categories are counted within the same screen.

6. The system 400 removes need for user to compare count results against historical count data. User can focus on performing a count and rely on the system to communicate areas of problem without forcing the user to match totals. The system 400 provides this communication during the count process or upon submission to the count sheet.

7. The system 400 permits a user to scroll through the performed count history and inventory baseline history throughout the surgery in time order on the same screen as performing the count. The user may verify count and baseline history entries directly on the same screen.

8. The system 400 increases patient safety and reduces hazards by increasing the accuracy of inventory counts. The system 400 marks data that is not desirable for the patient's safety. If any data is modified, an audit and reason is documented so that the information is stored and accessible in an audit trail.

9. The system 400 permits count functionality to be accessed on one user-friendly display interface, which increases usability and saves time. The user does not need to access multiple screens or dialogs within the web browser to complete a count throughout surgery. This is also true for reviewing history. Secondary dialogs are invoked from the count sheet screen when the user changes the inventory baseline or edits historical data. However, these are considered secondary tasks.

The system 400 and the method 500 are applicable to any field or area that requires a user to repetitively verify a quantity of items against a baseline quantity of items, and manage the baseline quantity of items at the same time. For example, the system 400 and the method 500 are usable when a retail store counts stock inventory on the shelf to verify that what is stored in a computer system matches shelf inventory. The system 400 and the method 500 are of particular use within an operating room management system to permit an instrument count workflow and document a count sheet that is able to be stored in an electronic patient medical record.

Hence, while the present invention has been described with reference to various illustrative examples thereof, it is not intended that the present invention be limited to these specific examples. Those skilled in the art will recognize that variations, modifications, and combinations of the disclosed subject matter can be made, without departing from the spirit and scope of the present invention, as set forth in the appended claims.

What is claimed is:

1. A system supporting validating a medical device and material inventory, comprising:
a display processor for initiating generation of data representing a user-interface display image presenting,
a pre-populated list of item types and associated quantities derived from a first source and
individual data fields corresponding to individual item types of said list, an individual data field being for receiving data representing a user entered quantity value representing a count of items of an individual item type; and
a data processor for comparing a user entered quantity value with a pre-populated quantity and for generating data representing a message in response to a difference determined by said comparison.

2. A system according to claim 1, wherein
said pre-populated list of item types and associated quantities is derived from a plurality of different sources including said first source.

3. A system according to claim 2, wherein
said plurality of different sources comprise two or more of, (a) a predetermined preferred list of item types to be used for a particular procedure by a healthcare worker, (b) a predetermined preferred list of item types to be used for operation of a particular medical device type, (c) a predetermined inventory list of item types, (d) a historical count list of item types previously entered by a user via a user interface display image derived from a repository and (e) an inventory list of item types imported from a second system.

4. A system according to claim 1, including
a communication processor for automatically updating inventory records in a repository in response to said user entered quantity value.

5. A system according to claim 1, including
a data acquisition unit for receiving data identifying items from a bar code scanner and for updating count values in said individual data fields in response to said received data identifying items.

6. A system according to claim 1, including
a data acquisition unit for receiving data identifying items by wireless communication and for updating count values in said individual data fields in response to said received data identifying items.

7. A system according to claim 6, wherein
said data identifying items received by wireless communication is derived from RFID tags on individual items.

8. A system according to claim 7, wherein
said RFID tags on said individual items are robust RFID tags able to withstand a sterilization procedure.

9. A system according to claim 6, wherein
said data identifying items received by wireless communication identifies items internal to a patient's anatomy.

10. A system according to claim 1, including
a repository including data representing a plurality of count lists of item types previously entered by a user via a user interface display image together with dates of creation of individual lists and
a navigation processor enabling a user to access and display selected count lists derived from said repository.

11. A system according to claim 1, wherein
said data processor compares a user entered quantity value with a pre-populated quantity value derived from a selected count list of said plurality of count lists of item types and for generating data representing a message in response to a difference determined by said comparison, in response to user command.

12. A system according to claim 1, including
a configuration processor enabling a user to associate a particular item type with a particular category and
a collation processor for automatically allocating received data representing said user entered quantity value representing said count of items of said individual item type to a particular category.

13. A system according to claim 1, including
a configuration processor enabling a user to associate a particular item type with a particular group comprising a subset of items and
a sorting processor for automatically entering received data representing quantity values representing counts of said subset of items in response to user entry of data representing a count of said particular item.

14. A system according to claim 1, wherein
said user interface display image include image elements enabling a user to enter data representing a user entered quantity value representing said count of items of said individual item type in a plurality of different ways.

15. A system according to claim 14, wherein
said plurality of different ways include at least two of, (a) by entry of a numerical value, (b) by selection of a decrement or increment button, (c) by entry of data indicating a group of a plurality of items and (d) by selection from a predetermined option list.

16. A system according to claim 1, wherein
said pre-populated list of item types and associated quantities is editable by a user and
said display image includes individual data fields for receiving data representing a user entered reason code for an edit.

17. A system according to claim 1, wherein
said pre-populated list of item types and associated quantities is editable by a user to indicate an item is broken and is now counted as two items.

18. A system according to claim 1, wherein
a user is able to enter an item in said pre-populated list by data entry via said displayed image.

19. A system according to claim 1, wherein
said pre-populated list of item types and associated quantities comprises a partial count of a particular item type.

20. A system according to claim 19, wherein
said particular item type is of sharp devices.

21. A method for validating a medical device and material inventory, comprising the activities of:
initiating generation of data representing a user interface display image presenting,
a pre-populated list of item types and associated quantities derived from a first source and
individual data fields corresponding to individual item types of said list, an individual data field being for receiving data representing a user entered quantity value representing a count of items of an individual item type; and
comparing a user entered quantity value with a pre-populated quantity and for generating data representing a message in response to a difference determined by said comparison.

22. A tangible storage medium incorporating machine-readable instructions for performing the activities of claim 21.

23. A system supporting validating a medical device and material inventory, comprising:
a repository for storing data representing a record of item types and associated quantities;
a data acquisition unit for receiving data identifying items by wireless communication and for updating count values of said data items in response to said received data identifying items;
a display processor for initiating generation of data representing a user interface display image presenting a list of item types and associated quantities and said associated count values updated by said data acquisition unit; and
a data processor for comparing said associated quantities and said associated count values, and for generating data, representing a message, in response to a difference determined by said comparison.

24. A system according to claim 23, wherein
said data identifying items received by wireless communication is derived from RFID tags on individual items.

25. A system according to claim 24, wherein
said RFID tags on said individual items are robust RFID tags able to withstand a sterilization procedure.
